SEQUENCE LISTING (27 sequences)

SEQ ID NO: 1 - DNA, 23 bp, Artificial Sequence
  PCR primer
atcgtccatgggtggcaagtggt

SEQ ID NO: 2 - DNA, 23 bp, Artificial Sequence
  PCR primer
cggctactagtgcagttcttgaa

SEQ ID NO: 3 - DNA, 24 bp, Artificial Sequence
  PCR primer
atcgtactagtgagccagtagatc

SEQ ID NO: 4 - DNA, 24 bp, Artificial Sequence
  PCR primer
cggctactagtttccttcgggcct

SEQ ID NO: 5 - DNA, 23 bp, Artificial Sequence
  PCR primer
atcgtccatggagccagtagatc

SEQ ID NO: 6 - DNA, 441 bp, Haemophilus influenzae
atggatccaaaaactttagccctttctttattagcagctggcgtactagcaggttgtagcagccattcatcaaatatggcgaatacccaaatgaaatcagacaaaatcattattgctcaccgtggtgctagcggttatttaccagagcatacgttagaatctaaagcacttgcttttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatggccacgtgtgatcagagctcaactagtggccaccatcaccatcaccattaatctaga

SEQ ID NO: 7 - protein, 144 aa, Haemophilus influenzae
MDPKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMATCDQSSTSGHHHHHH

SEQ ID NO: 8 - DNA, 648 bp, Human Immunodeficiency Virus
atgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgcactagtggccaccatcaccatcaccattaa

SEQ ID NO: 9 - protein, 215 aa, Human Immunodeficiency Virus
MGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCTSGHHHHHH

SEQ ID NO: 10 - DNA, 288 bp, Human Immunodeficiency Virus
atggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagacctcctcaaggcagtcagactcatcaagtttctctatcaaagcaacccacctcccaatcccgaggggacccgacaggcccgaaggaaactagtggccaccatcaccatcaccattaa

SEQ ID NO: 11 - protein, 95 aa, Human Immunodeficiency Virus
MEPVDPRLEPWKHPGSQPKTACTNCYCKKCCFHCQVCFITKALGISYGRKKRRQRRRPPQGSQTHQVSLSKQPTSQSRGDPTGPKETSGHHHHHH

SEQ ID NO: 12 - DNA, 909 bp, Human Immunodeficiency Virus
atgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgcactagtgagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagacctcctcaaggcagtcagactcatcaagtttctctatcaaagcaacccacctcccaatcccgaggggacccgacaggcccgaaggaaactagtggccaccatcaccatcaccattaa

SEQ ID NO: 13 - protein, 302 aa, Human Immunodeficiency Virus
MGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCTSEPVDPRLEPWKHPGSQPKTACTNCYCKKCCFHCQVCFITKALGISYGRKKRRQRRRPPQGSQTHQVSLSKQPTSQSRGDPTGPKETSGHHHHHH

SEQ ID NO: 14 - DNA, 1029 bp, Artificial Sequence
  Fusion construct
atggatccaaaaactttagccctttctttattagcagctggcgtactagcaggttgtagcagccattcatcaaatatggcgaatacccaaatgaaatcagacaaaatcattattgctcaccgtggtgctagcggttatttaccagagcatacgttagaatctaaagcacttgcttttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgcactagtggccaccatcaccatcaccattaa

SEQ ID NO: 15 - protein, 324 aa, Artificial Sequence
  Fusion construct
CSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCTSGHHHHHH

SEQ ID NO: 16 - DNA, 1290 bp, Artificial Sequence
  Fusion construct
atggatccaaaaactttagccctttctttattagcagctggcgtactagcaggttgtagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgcactagtgagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagacctcctcaaggcagtcagactcatcaagtttctctatcaaagcaacccacctcccaatcccgaggggacccgacaggcccgaaggaaactagtggccaccatcaccatcaccattaa

SEQ ID NO: 17 - protein, 411 aa, Artificial Sequence
  Fusion construct
CSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCTSEPVDPRLEPWKHPGSQPKTACTNCYCKKCCFHCQVCFITKALGISYGRKKRRQRRRPPQGSQTHQVSLSKQPTSQSRGDPTGPKETSGHHHHHH

SEQ ID NO: 18 - DNA, 981 bp, Artificial Sequence
  Fusion construct
atggatccaagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgcactagtggccaccatcaccatcaccattaa

SEQ ID NO: 19 - protein, 326 aa, Artificial Sequence
  Fusion construct
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCTSGHHHHHH

SEQ ID NO: 20 - DNA, 1242 bp, Artificial Sequence
  Fusion construct
atggatccaagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgcactagtgagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagacctcctcaaggcagtcagactcatcaagtttctctatcaaagcaacccacctcccaatcccgaggggacccgacaggcccgaaggaaactagtggccaccatcaccatcaccattaa

SEQ ID NO: 21 - protein, 413 aa, Artificial Sequence
  Fusion construct
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCTSEPVDPRLEPWKHPGSQPKTACTNCYCKKCCFHCQVCFITKALGISYGRKKRRQRRRPPQGSQTHQVSLSKQPTSQSRGDPTGPKETSGHHHHHH

SEQ ID NO: 22 - DNA, 288 bp, Human Immunodeficiency Virus
atggagccagtagatcctagactagagccctggaagcatccaggaagtcagcctaaaactgcttgtaccaattgctattgtaaaaagtgttgctttcattgccaagtttgtttcataacagctgccttaggcatctcctatggcaggaagaagcggagacagcgacgaagacctcctcaaggcagtcagactcatcaagtttctctatcaaagcaacccacctcccaatccaaaggggagccgacaggcccgaaggaaactagtggccaccatcaccatcaccattaa

SEQ ID NO: 23 - protein, 95 aa, Human Immunodeficiency Virus
MEPVDPRLEPWKHPGSQPKTACTNCYCKKCCFHCQVCFITAALGISYGRKKRRQRRRPPQGSQTHQVSLSKQPTSQSKGEPTGPKETSGHHHHHH

SEQ ID NO: 24 - DNA, 909 bp, Human Immunodeficiency Virus
atgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgcactagtgagccagtagatcctagactagagccctggaagcatccaggaagtcagcctaaaactgcttgtaccaattgctattgtaaaaagtgttgctttcattgccaagtttgtttcataacagctgccttaggcatctcctatggcaggaagaagcggagacagcgacgaagacctcctcaaggcagtcagactcatcaagtttctctatcaaagcaacccacctcccaatccaaaggggagccgacaggcccgaaggaaactagtggccaccatcaccatcaccattaa

SEQ ID NO: 25 - protein, 302 aa, Human Immunodeficiency Virus
MGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCTSEPVDPRLEPWKHPGSQPKTACTNCYCKKCCFHCQVCFITAALGISYGRKKRRQRRRPPQGSQTHQVSLSKQPTSQSKGEPTGPKETSGHHHHHH

SEQ ID NO: 26 - DNA, 57 bp, Artificial Sequence
  MCS polylinker
ttcgaaaccatggccgcggactagtggccaccatcaccatcaccattaacggaattc

SEQ ID NO: 27 - protein, 9 aa, Artificial Sequence
  MCS polylinker
TSGHHHHHH

(19) United States
(12) Patent Application Publication
(10) Pub. No.: US 2008/0089929 A1
(43) Pub. Date: Apr. 17, 2008

(54) FUSION PROTEINS COMPRISING HIV-1 TAT AND/OR NEF PROTEINS

(75) Inventors: Claudine BRUCK, Rixensart (BE); Stephane Andre Georges Godart, Rixensart (BE); Martine Marchand, Glabais (BE)

Correspondence Address: GLAXOSMITHKLINE;Corporate Intellectual Property - UW2220, P.O. Box 1539, King of Prussia, PA, 19406-0939, US

(73) Assignee: SmithKline Biologicals S.A.

(21) Appl. No.: 11/866,225

(22) Filed: Oct. 2, 2007

Related U.S. Application Data
(63) Continuation of application No. 10/687,060, filed on Oct. 16, 2003, now abandoned.
Application No. 10/687,060 is a continuation of application No. 09/509,239, filed on Mar. 23, 2000, now abandoned.

(30) Foreign Application Priority Data
Sep. 26, 1997 (GB) 9720585.0

(51) Int. Cl. A61K 9/127; A61K 38/16; A61P 37/00

(52) U.S. Cl. 424/450; 514/12

(57) ABSTRACT

The invention provides (a) an HIV Tat protein or derivative thereof linked to either (i) a fusion partner or (ii) an HIV Nef protein or derivative thereof; or (b) an HIV Nef protein or derivative thereof linked to either (i) a fusion partner or (ii) an HIV Tat protein or derivative thereof; or (c) an HIV Nef protein or derivative thereof linked to an HIV Tat protein or derivative thereof and a fusion partner. The invention further provides for a nucleic acid encoding such a protein and a host cell, such as Pichia Pastoris, transformed with the aforementioned nucleic acid.

### CROSS REFERENCE TO RELATED APPLICATION

This application is a continuation of U.S. patent application Ser. No. 10/687,060, filed 16 Oct. 2003, which is a continuation of U.S. patent application Ser. No. 09/509,239, filed 23 Mar. 2000, now abandoned, which is the National Stage of PCT/EP98/06040, filed 17 Sep. 1998. Each of these applications is incorporated herein by reference in its entirety. This application also claims benefit of the filing date of GB Patent Application Number GB 9720585.0, filed 26 Sep. 1997.

### SUMMARY OF THE INVENTION

The present invention relates to novel HIV protein constructs, to their use in medicine, to pharmaceutical compositions containing them and to methods of their manufacture.

In particular, the invention relates to fusion proteins comprising HIV-1 Tat and/or Nef proteins.

### BACKGROUND

HIV-1 is the primary cause of the acquired immune deficiency syndrome (AIDS) which is regarded as one of the world's major health problems. Although extensive research throughout the world, has been conducted to produce a vaccine, such efforts thus far, have not been successful.

Non-envelope proteins of HIV-1 have been described and include for example internal structural proteins such as the products of the gag and pol genes and, other non-structural proteins such as Rev, Nef, Vif and Tat (Greene et al., New England J. Med, 324, 5, 308 et seq (1991) and Bryant et al. (Ed. Pizzo), Pediatr. Infect. Dis. J., 11, 5, 390 et seq (1992).

HIV Nef and Tat proteins are early proteins, that is, they are expressed early in infection and in the absence of structural proteins.

### BRIEF DESCRIPTION OF THE DRAWINGS

FIG. 1A is a map of plasmid pRIT14586

FIG. 1B is the coding sequence of the first 127 amino acids of protein D and multiple doing site.

FIGS. 2A-H depict the DNA and amino acid sequences of Nef-His; Tat-His; Nef-Tat-His fusion and mutated Tat.

FIG. 3 is a map of plasmid pRIT14597.

FIG. 4 is an SDS-PAGE of Nef-Tat-his-fusion protein.

FIG. 5 is an SDS-PAGE of Nef-Tat-his fusion protein.

FIGS. 6A and B are bar graphs showing Tat-specific antibody titers and isotypes.

FIG. 7 is a pair of bar graphs showing the antigen-specific lymphoproliferative response to Tat and reduced Nef-Tat.

FIGS. 8A and B are line graphs illustrating cell binding mediated by Tat and Nef-Tat proteins.

FIGS. 9A and B are line graphs illustrating inhibition of cell growth by Tat and Nef-Tat proteins.

### DETAILED DESCRIPTION

According to the present invention there is provided a protein comprising
(a) an HIV Nef protein or derivative thereof linked to either (i) a fusion partner or (ii) an HIV Tat protein or derivative thereof; or
(b) an HIV Tat protein or derivative thereof linked to either (i) a fusion partner or (ii) an HIV Nef protein or derivative thereof; or
(c) an HIV Nef protein or derivative thereof linked to an HIV Tat protein or derivative thereof and a fusion partner.

By ‘fusion partner’ is meant any protein sequence that is not Tat or Nef.

Preferably the fusion partner is protein D or its' lipidated derivative Lipoprotein D, from Haemophilus influenzae B. In particular, it is preferred that the N-terminal third, i.e. approximately the first 100-130 amino acids are utilised. This is represented herein as Lipo D1/3. In a preferred embodiment of the invention the Nef protein or derivative thereof may be linked to the Tat protein or derivative thereof. Such Nef-Tat fusions may optionally also be linked to an fusion partner, such as protein D.

The fusion partner is normally linked to the N-terminus of the Nef or Tat protein.

Derivatives encompassed within the present invention include molecules with a C terminal Histidine tail which preferably comprises between 5-10 Histidine residues. Generally, a histidine tail containing n residues is represented herein as His (n). The presence of an histidine (or ‘His’) tail aids purification. More specifically, the invention provides proteins with the following structure

| | | |
|---|---|---|
| Lipo D 1/3 | Nef | His (₆) |
| Lipo D 1/3 | Nef-Tat | His (₆) |
| Prot D 1/3 | Nef | His (₆) |
| Prot D 1/3 | Nef-Tat | His (₆) |
| | Nef-Tat | His (₆) |

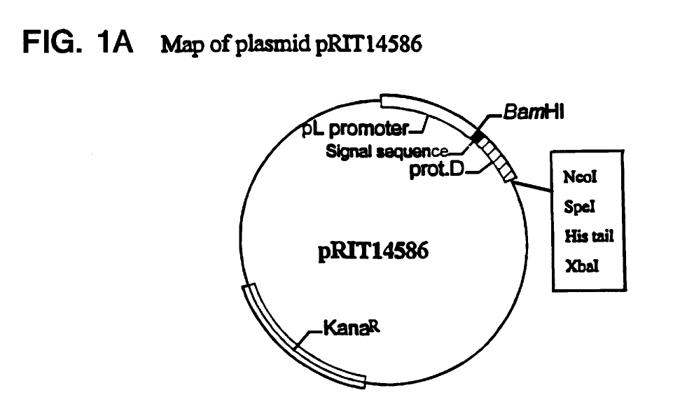

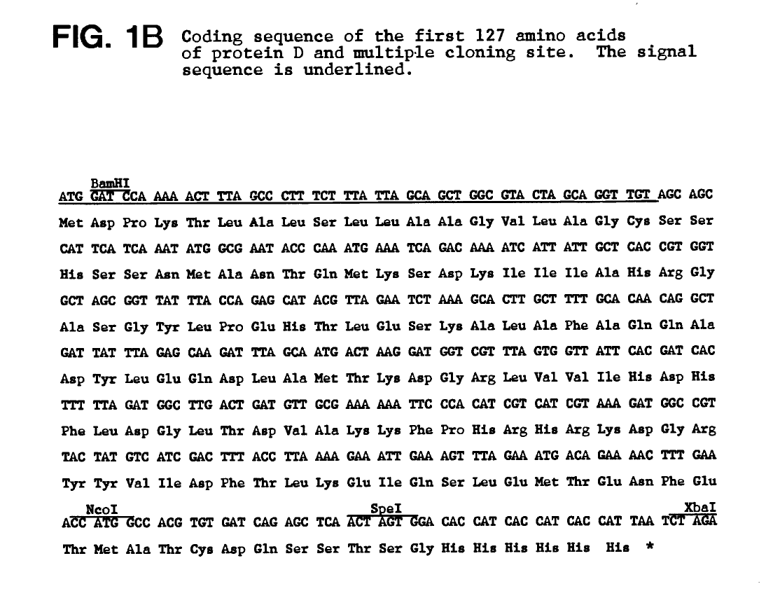

FIG. 1 provides the amino-acid (Seq. ID. No. 7) and DNA sequence (Seq. ID. No. 6) of the fusion partner for such constructs.

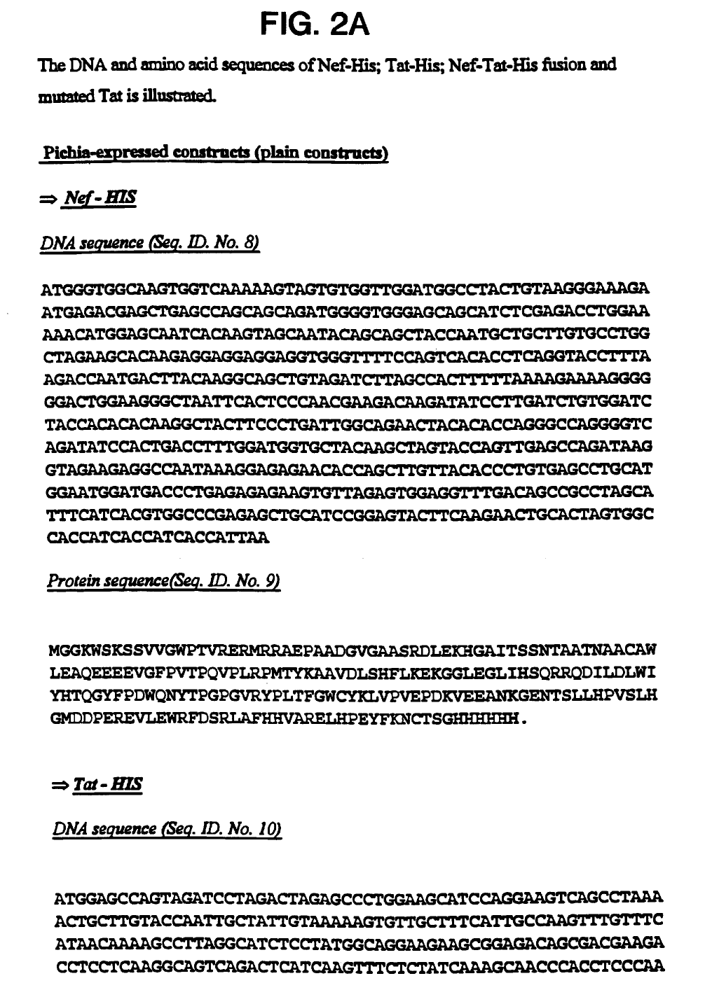

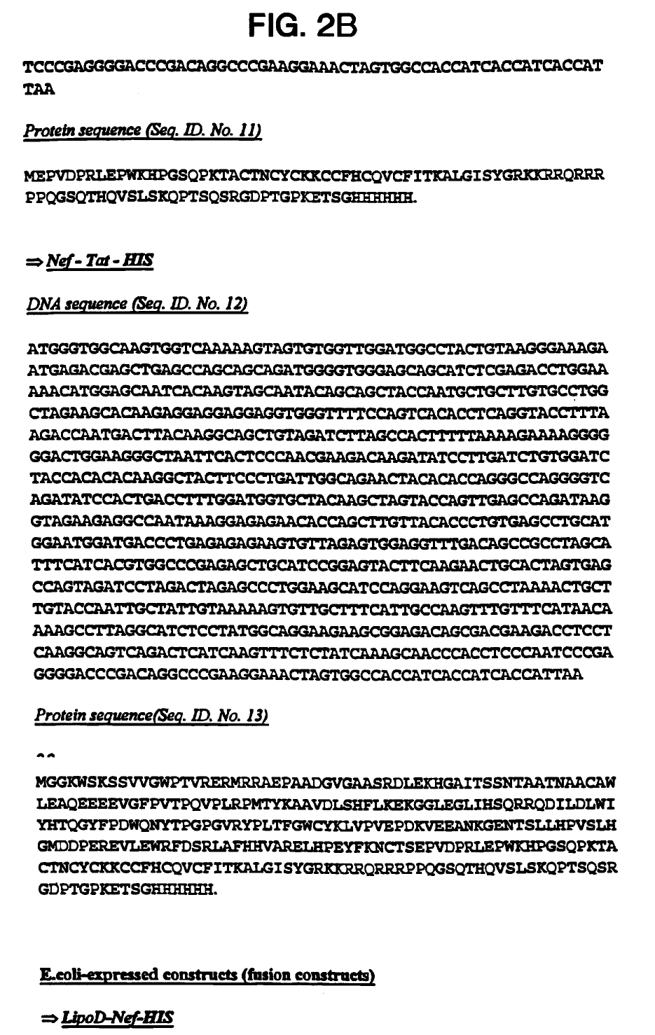

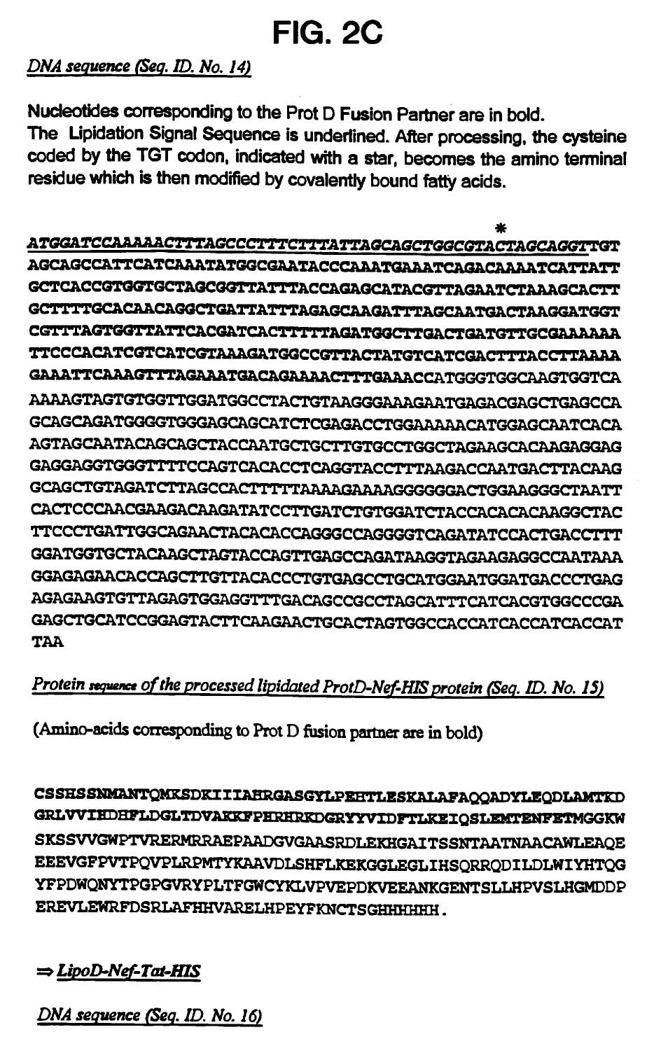

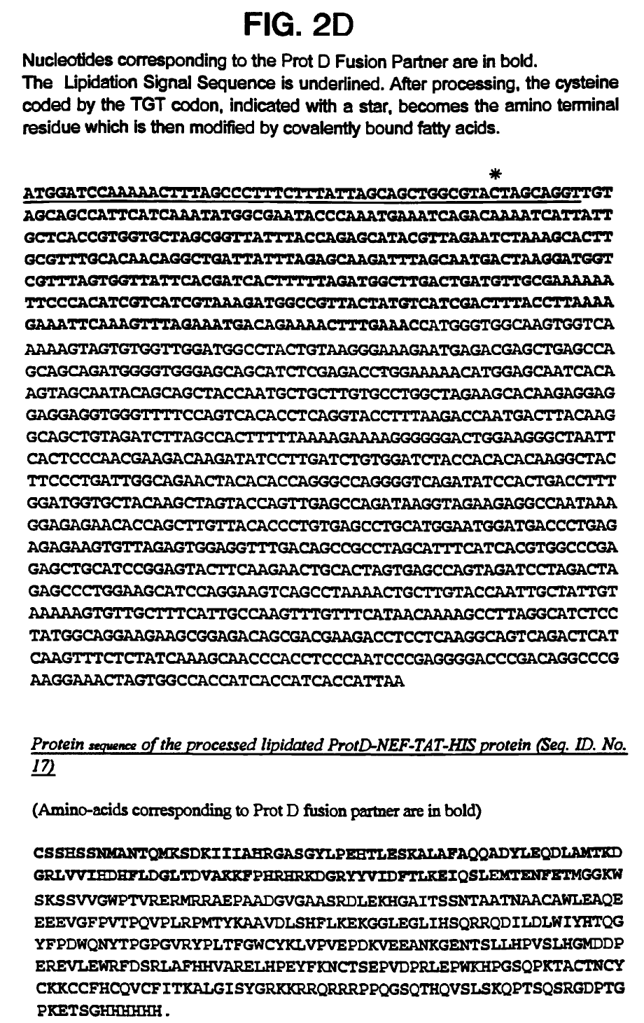

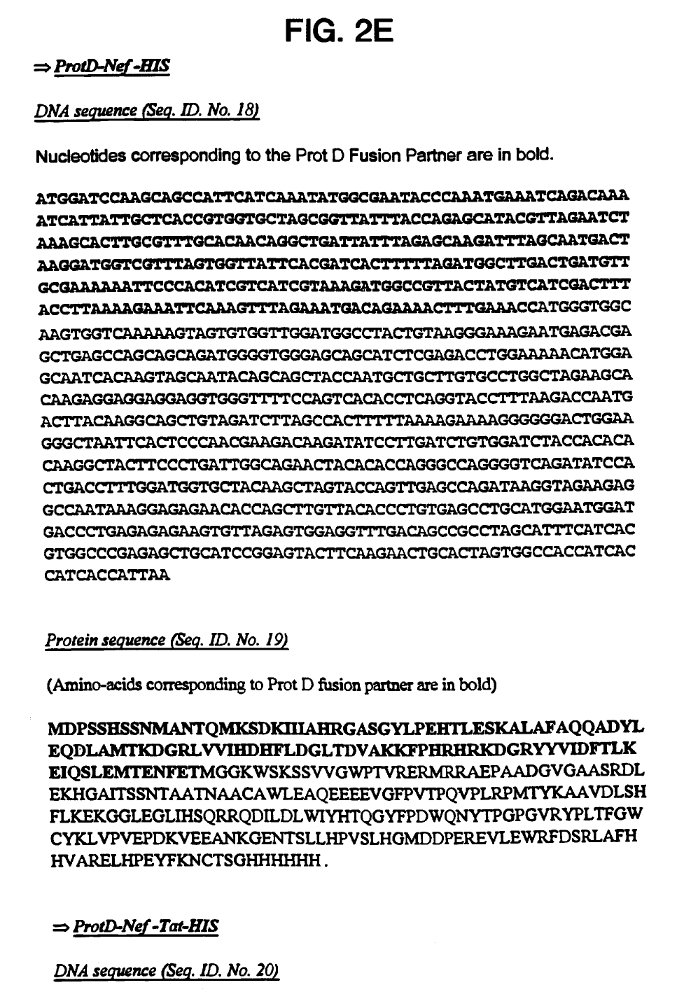

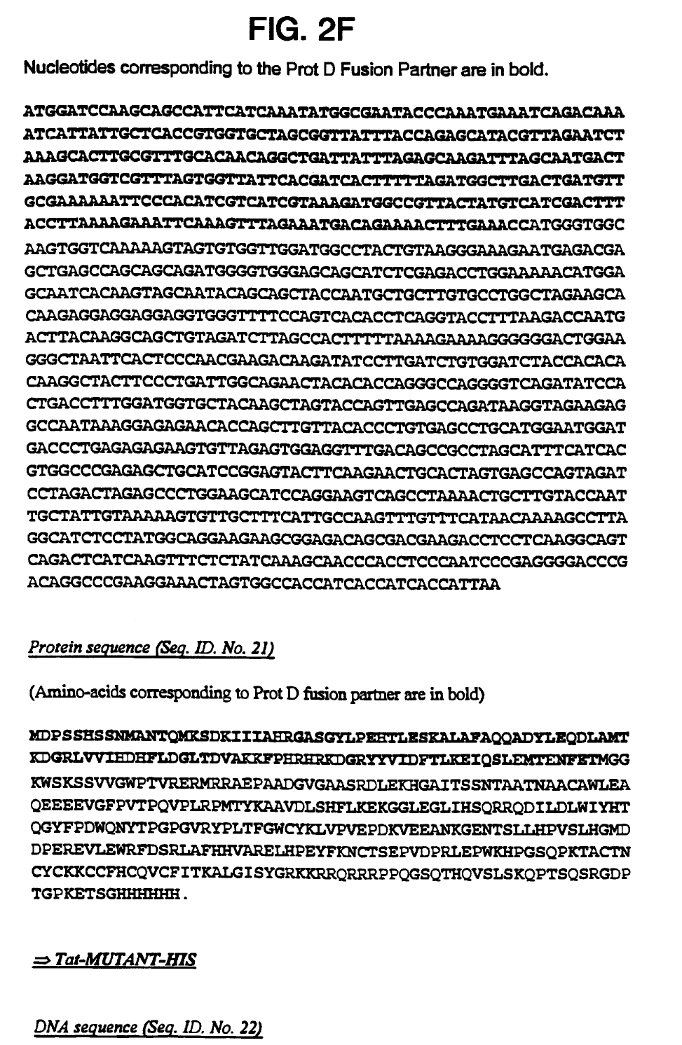

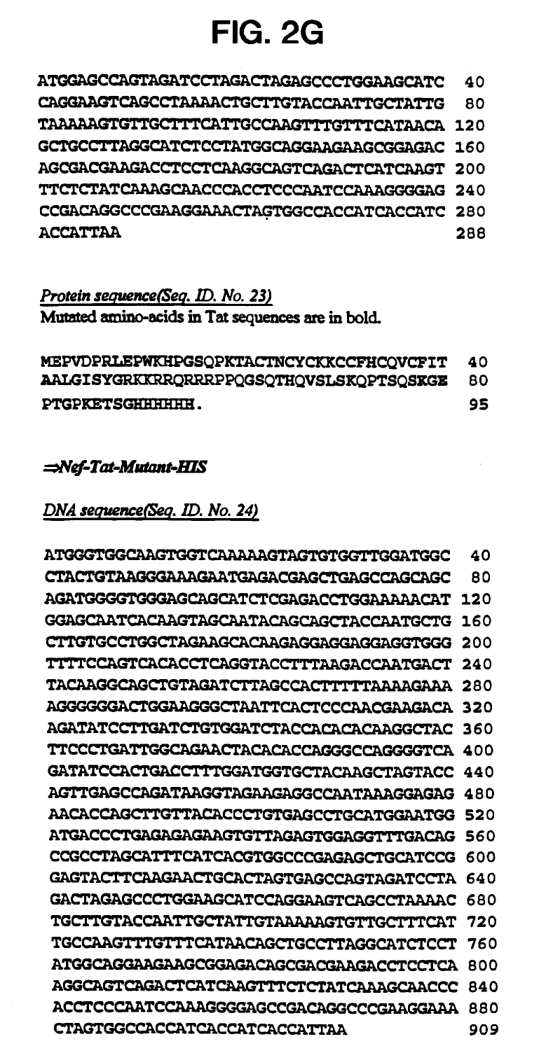

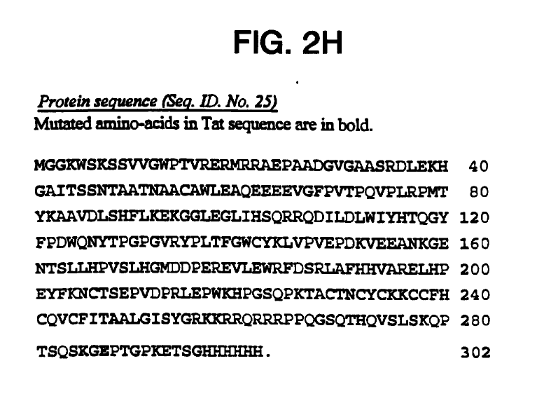

In a preferred embodiment the proteins are expressed with a Histidine tail comprising between 5 to 10 and preferably six Histidine residues. These are advantageous in aiding purification. Separate expression, in yeast (Saccharomyces cerevisiae), of Nef (Macreadie I. G. et al., 1993, Yeast 9 (6) 565-573) and Tat (Braddock M et al., 1989, Cell 58 (2) 269-79) has already been reported. Nef protein only is myristilated. The present invention provides for the first time the expression of Nef and Tat separately in a Pichia expression system (Nef-His and Tat-His constructs), and the successful expression of a fusion construct Nef-Tat-His. The DNA and amino acid sequences of representative Nef-His (Seq. ID. No.s 8 and 9), Tat-His (Seq. ID. No.s 10 and 11) and of Nef-Tat-His fusion proteins (Seq. ID. No.s 12 and 13) are set forth in FIG. 2.

Derivatives encompassed within the present invention also include mutated proteins. The term ‘mutated’ is used herein to mean a molecule which has undergone deletion, addition or substitution of one or more amino acids using well known techniques for site directed mutagenesis or any other conventional method.

A mutated Tat is illustrated in FIG. 2 (Seq. ID. No.s 22 and 23) as is a Nef-Tat Mutant-His (Seq. ID. No.s 24 and 25).

The present invention also provides a DNA encoding the proteins of the present invention. Such sequences can be inserted into a suitable expression vector and expressed in a suitable host.

A DNA sequence encoding the proteins of the present invention can be synthesized using standard DNA synthesis techniques, such as by enzymatic ligation as described by D. M. Roberts et al. in Biochemistry 1985, 24, 5090-5098, by chemical synthesis, by in vitro enzymatic polymerization, or by PCR technology utilising for example a heat stable polymerase, or by a combination of these techniques.

Enzymatic polymerisation of DNA may be carried out in vitro using a DNA polymerase such as DNA polymerase I (Klenow fragment) in an appropriate buffer containing the nucleoside triphosphates dATP, dCTP, dGTP and dTTP as required at a temperature of 10°-37° C., generally in a volume of 50 μl or less. Enzymatic ligation of DNA fragments may be carried out using a DNA ligase such as T4 DNA ligase in an appropriate buffer, such as 0.05M Tris (pH 7.4), 0.01M MgCl₂, 0.01M dithiothreitol, 1 mM spermidine, 1 mM ATP and 0.1 mg/ml bovine serum albumin, at a temperature of 4° C. to ambient, generally in a volume of 50 ml or less. The chemical synthesis of the DNA polymer or fragments may be carried out by conventional phosphotriester, phosphite or phosphoramidite chemistry, using solid phase techniques such as those described in ‘Chemical and Enzymatic Synthesis of Gene Fragments—A Laboratory Manual’ (ed. H. G. Gassen and A. Lang), Verlag Chemie, Weinheim (1982), or in other scientific publications, for example M. J. Gait, H. W. D. Matthes, M. Singh, B. S. Sproat, and R. C. Titmas, Nucleic Acids Research, 1982, 10, 6243; B. S. Sproat, and W. Bannwarth, Tetrahedron Letters, 1983, 24, 5771; M. D. Matteucci and M. H. Caruthers, Tetrahedron Letters, 1980, 21, 719; M. D. Matteucci and M. H. Caruthers, Journal of the American Chemical Society, 1981, 103, 3185; S. P. Adams et al, Journal of the American Chemical Society, 1983, 105, 661; N. D. Sinha, J. Biernat, J. McMannus, and H. Koester, Nucleic Acids Research, 1984, 12, 4539; and H. W. D. Matthes et al., EMBO Journal, 1984, 3, 801.

The invention also provides a process for preparing a protein of the invention, the process comprising the steps of:
i) preparing a replicable or integrating expression vector capable, in a host cell, of expressing a DNA polymer comprising a nucleotide sequence that encodes the protein or a derivative thereof
ii) transforming a host cell with said vector
iii) culturing said transformed host cell under conditions permitting expression of said DNA polymer to produce said protein; and
iv) recovering said protein

The process of the invention may be performed by conventional recombinant techniques such as described in Maniatis et al., Molecular Cloning—A Laboratory Manual; Cold Spring Harbor, 1982-1989.

The term ‘transforming’ is used herein to mean the introduction of foreign DNA into a host cell. This can be achieved for example by transformation, transfection or infection with an appropriate plasmid or viral vector using e.g. conventional techniques as described in Genetic Engineering; Eds. S. M. Kingsman and A. J. Kingsman; Blackwell Scientific Publications; Oxford, England, 1988. The term ‘transformed’ or ‘transformant’ will hereafter apply to the resulting host cell containing and expressing the foreign gene of interest.

The expression vectors are novel and also form part of the invention.

The replicable expression vectors may be prepared in accordance with the invention, by cleaving a vector compatible with the host cell to provide a linear DNA segment having an intact replicon, and combining said linear segment with one or more DNA molecules which, together with said linear segment encode the desired product, such as the DNA polymer encoding the protein of the invention, or derivative thereof, under ligating conditions.

Thus, the DNA polymer may be preformed or formed during the construction of the vector, as desired.

The choice of vector will be determined in part by the host cell, which may be prokaryotic or eukaryotic but preferably is E. coli or yeast. Suitable vectors include plasmids, bacteriophages, cosmids and recombinant viruses.

The preparation of the replicable expression vector may be carried out conventionally with appropriate enzymes for restriction, polymerisation and ligation of the DNA, by procedures described in, for example, Maniatis et al. cited above.

The recombinant host cell is prepared, in accordance with the invention, by transforming a host cell with a replicable expression vector of the invention under transforming conditions. Suitable transforming conditions are conventional and are described in, for example, Maniatis et al. cited above, or “DNA Cloning” Vol. II, D. M. Glover ed., IRL Press Ltd, 1985.

The choice of transforming conditions is determined by the host cell. Thus, a bacterial host such as E. coli may be treated with a solution of CaCl₂ (Cohen et al., Proc. Nat. Acad. Sci., 1973, 69, 2110) or with a solution comprising a mixture of RbCl, MnCl₂, potassium acetate and glycerol, and then with 3-[N-morpholino]-propane-sulphonic acid, RbCl and glycerol. Mammalian cells in culture may be transformed by calcium co-precipitation of the vector DNA onto the cells. The invention also extends to a host cell transformed with a replicable expression vector of the invention.

Culturing the transformed host cell under conditions permitting expression of the DNA polymer is carried out conventionally, as described in, for example, Maniatis et al. and “DNA Cloning” cited above. Thus, preferably the cell is supplied with nutrient and cultured at a temperature below 50° C.

The product is recovered by conventional methods according to the host cell. Thus, where the host cell is bacterial, such as E. coli—or yeast such as Pichia; it may be lysed physically, chemically or enzymatically and the protein product isolated from the resulting lysate. Where the host cell is mammalian, the product may generally be isolated from the nutrient medium or from cell free extracts. Conventional protein isolation techniques include selective precipitation, adsorption chromatography, and affinity chromatography including a monoclonal antibody affinity column.

For proteins of the present invention provided with Histidine tails, purification can easily be achieved by the use of a metal ion affinity column. In a preferred embodiment, the protein is further purified by subjecting it to cation ion exchange chromatography and/or Gel filtration chromatography. The protein is then sterilised by passing through a 0.22 μm membrane.

The proteins of the invention can then be formulated as a vaccine, or the Histidine residues enzymatically cleared.

The proteins of the present invention are provided preferably at least 80% pure more preferably 90% pure as visualised by SDS PAGE. Preferably the proteins appear as a single band by SDS PAGE.

The present invention also provides pharmaceutical composition comprising a protein of the present invention in a pharmaceutically acceptable excipient.

Vaccine preparation is generally described in New Trends and Developments in Vaccines, Voller et al. (eds.), University Park Press, Baltimore, Md., 1978. Encapsulation within liposomes is described by Fullerton, U.S. Pat. No. 4,235,877.

The proteins of the present invention are preferably adjuvanted in the vaccine formulation of the invention. Suitable adjuvants include an aluminium salt such as aluminium hydroxide gel (alum) or aluminium phosphate, but may also be a salt of calcium, iron or zinc, or may be an insoluble suspension of acylated tyrosine, or acylated sugars, cationically or anionically derivatised polysaccharides, or polyphosphazenes.

In the formulation of the inventions it is preferred that the adjuvant composition induces a preferential TH1 response. Suitable adjuvant systems include, for example, a combination of monophosphoryl lipid A or derivative thereof, preferably 3-de-O-acylated monophosphoryl lipid A (3D-MPL) together with an aluminium salt.

An enhanced system involves the combination of a monophosphoryl lipid A and a saponin derivative particularly the combination of QS21 and 3D-MPL as disclosed in WO 94/00153, or a less reactogenic composition where the QS21 is quenched with cholesterol as disclosed in WO 96/33739.

A particularly potent adjuvant formulation involving QS21, 3D-MPL & tocopherol in an oil in water emulsion is described in WO 95/17210 and is a preferred formulation.

Accordingly in one embodiment of the present invention there is provided a vaccine comprising a protein according to the invention adjuvanted with a monophosphoryl lipid A or derivative thereof, especially 3D-MPL.

Preferably the vaccine additionally comprises a saponin, more preferably QS21.

Preferably the formulation additional comprises an oil in water emulsion and tocopherol. The present invention also provides a method for producing a vaccine formulation comprising mixing a protein of the present invention together with a pharmaceutically acceptable excipient, such as 3D-MPL.

The vaccine of the present invention may additional comprise further HIV proteins, such as the envelope glycoprotein gp160 or its derivative gp120.

In another aspect, the invention relates to an HIV Nef or an HIV Tat protein or derivative thereof expressed in Pichia pastoris.

The invention will be further described by reference to the following examples:

### EXAMPLES

### General

Nef and Tat proteins, two regulatory proteins encoded by the human immunodeficiency virus (HIV-1) were produced in E. coli and in the methylotrophic yeast Pichia pastoris.

The nef gene from the Bru/Lai isolate (Cell 40: 9-17, 1985) was selected for these constructs since this gene is among those that are most closely related to the consensus Nef.

The starting material for the Bru/Lai nef gene was a 1170 bp DNA fragment cloned on the mammalian expression vector pcDNA3 (pcDNA3/nef).

The tat gene originates from the BH10 molecular clone. This gene was received as an HTLV III cDNA clone named pCV1 and described in Science, 229, p 69-73, 1985.

1. Expression of HIV-1 nef and tat Sequences in E. coli.

Sequences encoding the Nef protein as well as a fusion of nef and tat sequences were placed in plasmids vectors: pRIT14586 and pRIT14589 (see FIG. 1).

Nef and the Nef-Tat fusion were produced as fusion proteins using as fusion partner a part of the protein D. Protein D is an immunoglobulin D binding protein exposed at the surface of the gram-negative bacterium Haemophilus influenzae.

pRIT14586 contains, under the control of a λPL promoter, a DNA sequence derived from the bacterium Haemophilus influenzae which codes for the first 127 amino acids of the protein D (Infect. Immun. 60: 1336-1342, 1992), immediately followed by a multiple cloning site region plus a DNA sequence coding for one glycine, 6 histidines residues and a stop codon (FIG. 1A).

This vector is designed to express a processed lipidated His tailed fusion protein (LipoD fusion protein). The fusion protein is synthesised as a precursor with an 18 amino acid residues long signal sequence and after processing, the cysteine at position 19 in the precursor molecule becomes the amino terminal residue which is then modified by covalently bound fatty acids (FIG. 1B).

pRIT14589 is almost identical to pRIT14586 except that the protD derived sequence starts immediately after the cysteine 19 codon.

Expression from this vector results in a His tailed, non lipidated fusion protein (Prot D fusion protein).

Four constructs were made: LipoD-nef-His, LipoD-nef-tat-His, ProtD-nef-His, and ProtD-nef-tat-His.

The first two constructs were made using the expression vector pRIT14586, the last two constructs used pRIT14589.

1.1 Construction of the Recombinant Strain ECLD-N1 Producing the LipoD-Nef-His Fusion Protein.

1.1.1 Construction of the LipoD-nef-His Expression Plasmid pRIT14595

The nef gene (Bru/Lai isolate) was amplified by PCR from pcDNA3/Nef plasmid with primers 01 and 02.

```
PRIMER 01 (Seq ID NO 1):
                NcoI
5′ATCGTCCATG.GGT.GGC.AAG.TGG.T 3′
PRIMER 02 (Seq ID NO 2):
                      SpeI
5′ CGGCTACTAGTGCAGTTCTTGAA 3′
```

The nef DNA region amplified starts at nucleotide 8357 and terminates at nucleotide 8971 (Cell, 40: 9-17, 1985).

An NcoI restriction site (which carries the ATG codon of the nef gene) was introduced at the 5′ end of the PCR fragment while a SpeI site was introduced at the 3′ end.

The PCR fragment obtained and the expression plasmid pRIT14586 were both restricted by NcoI and SpeI, purified on an agarose gel, ligated and transformed in the appropriate E. coli host cell, strain AR58. This strain is a cryptic λ lysogen derived from N99 that is galE::Tn10, Δ-8 (chlD-pgl), Δ-H1 (cro-chlA), N⁺, and cI857.

The resulting recombinant plasmid received, after verification of the nef amplified region by automatic sequencing, (see section 1.1.2 below) the pRIT14595 denomination.

1.1.2 Selection of Transformants of E. Coli Strain AR58 with pRIT14595

When transformed in AR58 E. coli host strain, the recombinant plasmid directs the heat-inducible production of the heterologous protein.

Heat inducible protein production of several recombinant lipoD-Nef-His transformants was analysed by Coomassie Blue stained SDS-PAGE. All the transformants analysed showed an heat inducible heterologous protein production. The abundance of the recombinant Lipo D-Nef-Tat-His fusion protein was estimated at 10% of total protein.

One of the transformants was selected and given the laboratory accession number ECLD-N1.

The recombinant plasmid was reisolated from strain ECLD-N1, and the sequence of the nef-His coding region was confirmed by automated sequencing. This plasmid received the official designation pRIT14595.

The fully processed and acylated recombinant Lipo D-nef-His fusion protein produced by strain ECLD-N1 is composed of:
Fatty acids
109 a.a. of proteinD (starting at a.a.19 and extending to a.a.127).
A methionine, created by the use of NcoI cloning site of pRIT14586 (FIG. 1).
205a.a. of Nef protein (starting at a.a.2 and extending to a.a.206).
A threonine and a serine created by the cloning procedure (cloning at SpeI site of pRIT14586).
One glycine and six histidines.
   1.2 Construction of Recombinant Strain ECD-N1 Producing Prot D-Nef-His Fusion Protein.

Construction of expression plasmid pRIT14600 encoding the Prot D-Nef-His fusion protein was identical to the plasmid construction described in example 1.1.1 with the exception that pRIT14589 was used as receptor plasmid for the PCR amplified nef fragment.

E. coli AR58 strain was transformed with pRIT14600 and transformants were analysed as described in example 1.1.2. The transformant selected received laboratory accession number ECD-N1.

1.3 Construction of Recombinant Strain ECLD-NT6 Producing the Lipo D-Nef-Tat-His Fusion Protein.

1.3.1 Construction of the Lipo D-Nef-Tat-His Expression Plasmid pRIT14596

The tat gene (BH10 isolate) was amplified by PCR from a derivative of the pCV1 plasmid with primers 03 and 04. SpeI restriction sites were introduced at both ends of the PCR fragment.

```
PRIMER 03 (Seq ID NO 3):
                      SpeI
5′ ATCGTACTAGT.GAG.CCA.GTA.GAT.C 3′
PRIMER 04 (Seq ID NO 4):
                      SpeI
5′ CGGCTACTAGTTTCCTTCGGGCCT 3′
```

The nucleotide sequence of the amplified tat gene is illustrated in the pCV1 clone (Science 229: 69-73, 1985) and covers nucleotide 5414 till nucleotide 7998.

The PCR fragment obtained and the plasmid pRIT14595 (expressing lipoD-Nef-His protein) were both digested by SpeI restriction enzyme, purified on an agarose gel, ligated and transformed in competent AR58 cells. The resulting recombinant plasmid received, after verification of the tat amplified sequence by automatic sequencing (see section 1.3.2 below), the pRIT14596 denomination.

1.3.2 Selection of Transformants of Strain AR58 with pRIT14596

Transformants were grown, heat induced and their proteins were analysed by Coomassie Blue stained gels. The production level of the recombinant protein was estimated at 1% of total protein. One recombinant strain was selected and received the laboratory denomination ECLD-NT6.

The lipoD-nef-tat-His recombinant plasmid was reisolated from ECLD-NT6 strain, sequenced and received the official designation pRIT14596.

The fully processed and acylated recombinant Lipo D-Nef-Tat-His fusion protein produced by strain ECLD-N6 is composed of:
Fatty acids
109 a.a. of proteinD (starting at a.a.19 and extending to a.a.127).
A methionine, created by the use of NcoI cloning site of pRIT14586.
205a.a. of the Nef protein (starting at a.a.2 and extending to a.a.206)
A threonine and a serine created by the cloning procedure
85a.a. of the Tat protein (starting at a.a.2 and extending to a.a.86)
A threonine and a serine introduced by cloning procedure
One glycine and six histidines.
   1.4 Construction of Recombinant Strain ECD-NT1 Producing Prot D-Nef-Tat-His Fusion Protein.

Construction of expression plasmid pRIT14601 encoding the Prot D-Nef-Tat-His fusion protein was identical to the plasmid construction described in example 1.3.1 with the exception that pRIT14600 was used as receptor plasmid for the PCR amplified nef fragment.

E. coli AR58 strain was transformed with pRIT14601 and transformants were analysed as described previously. The transformant selected received laboratory accession number ECD-NT1.

2. Expression of HIV-1 nef and tat Sequences in Pichia pastoris.

Nef protein, Tat protein and the fusion Nef-Tat were expressed in the methylotrophic yeast Pichia pastoris under the control of the inducible alcohol oxidase (AOX1) promoter.

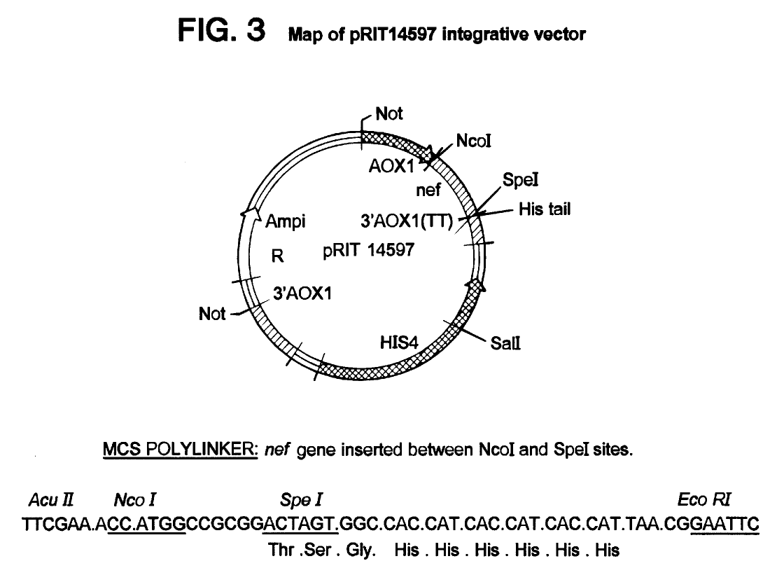

To express these HIV-1 genes a modified version of the integrative vector PHIL-D2 (INVITROGEN) was used. This vector was modified in such a way that expression of heterologous protein starts immediately after the native ATG codon of the AOX1 gene and will produce recombinant protein with a tail of one glycine and six histidines residues. This PHIL-D2-MOD vector was constructed by cloning an oligonucleotide linker between the adjacent AsuII and EcoRI sites of PHIL-D2 vector (see FIG. 3). In addition to the His tail, this linker carries NcoI, SpeI and XbaI restriction sites between which nef, tat and nef-tat fusion were inserted.

2.1 Construction of the Integrative Vectors pRIT14597 (Encoding Nef-His Protein), pRIT14598 (Encoding Tat-His Protein) and pRIT14599 (Encoding Fusion Nef-Tat-His).

The nef gene was amplified by PCR from the pcDNA3/Nef plasmid with primers 01 and 02 (see section 1.1.1 construction of pRIT14595). The PCR fragment obtained and the integrative PHIL-D2-MOD vector were both restricted by NcoI and SpeI, purified on agarose gel and ligated to create the integrative plasmid pRIT14597 (see FIG. 3).

The tat gene was amplified by PCR from a derivative of the pCV1 plasmid with primers 05 and 04 (see section 1.3.1 construction of pRIT14596):

PRIMER 05 (Seq ID NO 5):
NcoI
5′ATCGTCCATGGAGCCAGTAGATC 3′

An NcoI restriction site was introduced at the 5′ end of the PCR fragment while a SpeI site was introduced at the 3′ end with primer 04. The PCR fragment obtained and the PHIL-D2-MOD vector were both restricted by NcoI and SpeI, purified on agarose gel and ligated to create the integrative plasmid pRIT14598.

To construct pRIT14599, a 910 bp DNA fragment corresponding to the nef-tat-His coding sequence was ligated between the EcoRI blunted (T4 polymerase) and NcoI sites of the PHIL-D2-MOD vector. The nef-tat-His coding fragment was obtained by XbaI blunted (T4 polymerase) and NcoI digestions of pRIT14596.

2.2 Transformation of Pichia pastoris Strain GS115(his4).

To obtain Pichia pastoris strains expressing Nef-His, Tat-His and the fusion Nef-Tat-His, strain GS115 was transformed with linear NotI fragments carrying the respective expression cassettes plus the HIS4 gene to complement his4 in the host genome. Transformation of GS115 with NotI-linear fragments favors recombination at the AOXI locus.

Multicopy integrant clones were selected by quantitative dot blot analysis and the type of integration, insertion (Mut⁺phenotype) or transplacement (Mut^{s}phenotype), was determined.

From each transformation, one transformant showing a high production level for the recombinant protein was selected:

Strain Y1738 (Mut⁺ phenotype) producing the recombinant Nef-His protein, a myristylated 215 amino acids protein which is composed of:
Myristic acid
A methionine, created by the use of NcoI cloning site of PHIL-D2-MOD vector
205 a.a. of Nef protein (starting at a.a.2 and extending to a.a.206)
A threonine and a serine created by the cloning procedure (cloning at SpeI site of PHIL-D2-MOD vector.
One glycine and six histidines.

Strain Y1739 (Mut⁺ phenotype) producing the Tat-His protein, a 95 amino acid protein which is composed of:
A methionine created by the use of NcoI cloning site
85 a.a. of the Tat protein (starting at a.a.2 and extending to a.a.86)
A threonine and a serine introduced by cloning procedure
One glycine and six histidines

Strain Y1737 (Mut^{s} phenotype) producing the recombinant Nef-Tat-His fusion protein, a myristylated 302 amino acids protein which is composed of:
Myristic acid
A methionine, created by the use of NcoI cloning site
205a.a. of Nef protein (starting at a.a.2 and extending to a.a.206)
A threonine and a serine created by the cloning procedure
85a.a. of the Tat protein (starting at a.a.2 and extending to a.a.86)
A threonine and a serine introduced by the cloning procedure
One glycine and six histidines
   3. Expression of HIV-1 Tat-Mutant in Pichia pastoris

As well as a Nef-Tat mutant fusion protein, a mutant recombinant Tat protein has also been expressed. The mutant Tat protein must be biologically inactive while maintaining its immunogenic epitopes.

A double mutant tat gene, constructed by D. Clements (Tulane University) was selected for these constructs.

This tat gene (originates from BH10 molecular clone) bears mutations in the active site region (Lys41→Ala) and in RGD motif (Arg78→Lys and Asp8→Glu) (Virology 235: 48-64, 1997).

The mutant tat gene was received as a cDNA fragment subcloned between the EcoRI and HindIII sites within a CMV expression plasmid (pCMVLys41/KGE)

3.1 Construction of the Integrative Vectors pRIT14912 (Encoding Tat Mutant-His Protein) and pRIT14913 (Encoding Fusion Nef-Tat Mutant-His).

The tat mutant gene was amplified by PCR from the pCMVLys41/KGE plasmid with primers 05 and 04 (see section 2.1 construction of pRIT14598)

An NcoI restriction site was introduced at the 5′ end of the PCR fragment while a SpeI site was introduced at the 3′ end with primer 04. The PCR fragment obtained and the PHIL-D2-MOD vector were both restricted by NcoI and SpeI, purified on agarose gel and ligated to create the integrative plasmid pRIT14912

To construct pRIT14913, the tat mutant gene was amplified by PCR from the pCMVLys41/KGE plasmid with primers 03 and 04 (see section 1.3.1 construction of pRIT14596).

The PCR fragment obtained and the plasmid pRIT14597 (expressing Nef-His protein) were both digested by SpeI restriction enzyme, purified on agarose gel and ligated to create the integrative plasmid pRIT14913

3.2 Transformation of Pichia pastoris Strain GS115.

Pichia pastoris strains expressing Tat mutant-His protein and the fusion Nef-Tat mutant-His were obtained, by applying integration and recombinant strain selection strategies previously described in section 2.2.

Two recombinant strains producing Tat mutant-His protein, a 95 amino-acids protein, were selected: Y1775 (Mut⁺ phenotype) and Y1776 (Mut^{s} phenotype).

One recombinant strain expressing Nef-Tat mutant-His fusion protein, a 302 amino-acids protein was selected: Y1774 (Mut⁺ phenotype).

4. Purification of Nef-Tat-His Fusion Protein (Pichia pastoris)

The purification scheme has been developed from 146 g of recombinant Pichia pastoris cells (wet weight) or 2 L Dyno-mill homogenate OD 55. The chromatographic steps are performed at room temperature. Between steps, Nef-Tat positive fractions are kept overnight in the cold room (+4° C.); for longer time, samples are frozen at −20° C.

| | |
|---|---|
| 146 g of Pichia pastoris | |
| cells | |
| ↓ | |
| Homogenization | Buffer: 2 L 50 mM PO₄ pH 7.0 |
| | final OD: 50 |
| ↓ | |
| Dyno-mill disruption | |
| (4 passes) | |
| ↓ | |
| Centrifugation | JA10 rotor/9500 rpm/30 min/ |
| | room temperature |
| ↓ | |
| Dyno-mill Pellet | |
| ↓ | |
| Wash | Buffer: +2 L 10 mM PO₄ pH 7.5 - |
| (1 h-4° C.) | 150 mM - NaCl 0.5% empigen |
| ↓ | |
| Centrifugation | JA10 rotor/9500 rpm/30 min/ |
| | room temperature |
| ↓ | |
| Pellet | |
| ↓ | |
| Solubilisation | Buffer: +660 ml 10 mM PO₄ pH |
| (O/N - 4° C.) | 7.5 - 150 mM NaCl - 4.0M GuHCl |
| ↓ | |
| Reduction | +0.2M 2-mercaptoethanesulfonic |
| (4 H - room temperature - | acid, sodium salt (powder |
| in the dark) | addition)/pH adjusted to 7.5 |
| | (with 0.5M NaOH solution) before |
| | incubation |
| ↓ | |
| Carboxymethylation | +0.25M Iodoacetamid (powder |
| (½ h - room | addition)/pH adjusted to 7.5 |
| temperature - in the dark) | (with 0.5M NaOH solution) before |
| | incubation |
| ↓ | |
| Immobilized metal ion | Equilibration buffer: 10 mM PO₄ |
| affinity chromatography | pH 7.5 - 150 mM NaCl - 4.0M |
| on Ni⁺⁺-NTA-Agarose | GuHCl |
| (Qiagen - 30 ml of resin) | |
| | Washing buffer: 1) Equilibration |
| | buffer |
| | 2) 10 m MPO₄ |
| | pH 7.5 - 150 mM NaCl - 6M Urea |
| | 3) 10 mM PO₄ |
| | pH 7.5 - 150 mM NaCl - 6M Urea - |
| | 25 mM Imidazol |
| | Elution buffer: 10 mM PO₄ pH 7.5 - |
| | 150 mM NaCl - 6M Urea - 0.5M |
| | Imidazol |
| ↓ | |
| Dilution | Down to an ionic strength of 18 |
| | mS/cm² |
| | Dilution buffer: 10 mM PO₄ pH |
| | 7.5 - 6M Urea |
| ↓ | |
| Cation exchange chromatog- | Equilibration buffer: 10 mM PO₄ |
| raphy on SP Sepharose FF | pH 7.5 - 150 mM NaCl - 6.0M |
| (Pharmacia - 30 ml of | Urea |
| resin) | |
| | Washing buffer: 1) Equilibration |
| | buffer |
| | 2) 10 mM PO₄ |
| | pH 7.5 - 250 mM NaCl - 6M Urea |
| | Elution buffer: 10 mM Borate pH |
| | 9.0 - 2M NaCl - 6M Urea |
| ↓ | |
| Concentration | up to 5 mg/ml |
| | 10 kDa Omega membrane(Filtron) |
| ↓ | |
| Gel filtration chromatog- | Elution buffer: 10 mM PO₄ pH 7.5 - |
| raphy on Superdex200 XK | 150 mM NaCl - 6M Urea |
| 16/60 (Pharmacia - 120 | 5 ml of sample/injection → 5 |
| ml of resin) | injections |
| ↓ | |
| Dialysis | Buffer: 10 mM PO₄ pH 6.8 - |
| (O/N - 4° C.) | 150 mM NaCl - 0.5M Arginin* |
| ↓ | |
| Sterile filtration | Millex GV 0.22 μm |

*ratio: 0.5M Arginin for a protein concentration of 1600 μg/ml.

Purity

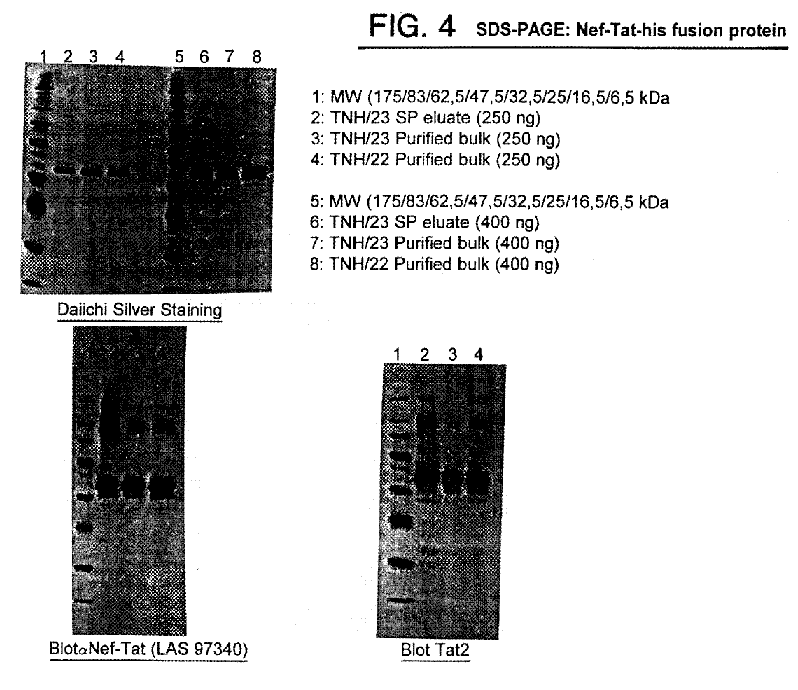

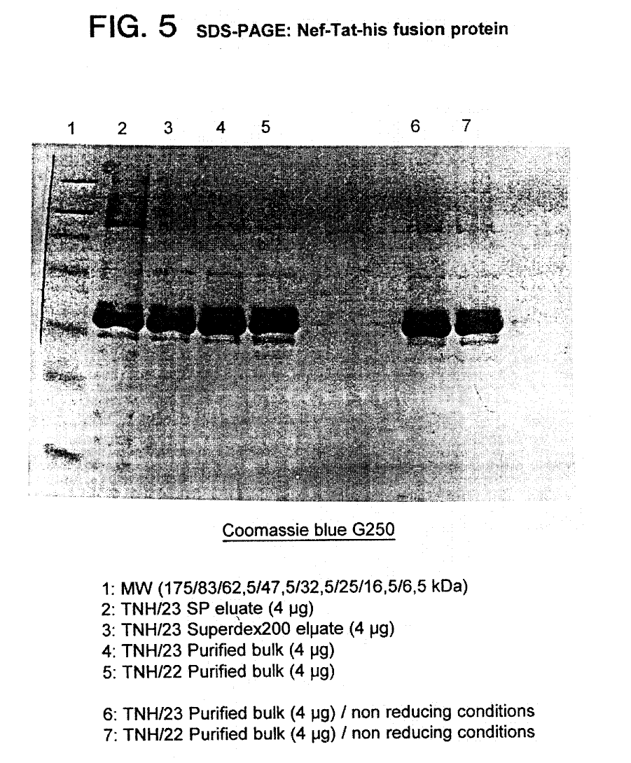

The level of purity as estimated by SDS-PAGE is shown in FIG. 4 by Daiichi Silver Staining and in FIG. 5 by Coomassie blue G250.

| | |
|---|---|
| After Superdex200 step: | >95% |
| After dialysis and sterile filtration steps: | >95% |

Recovery

51 mg of Nef-Tat-his protein are purified from 146 g of recombinant Pichia pastoris cells (=2 L of Dyno-mill homogenate OD 55)

5. Vaccine Preparation

A vaccine prepared in accordance with the invention comprises the expression product of a DNA recombinant encoding an antigen as exemplified in example 1 or 2 and as adjuvant, the formulation comprising a mixture of 3 de-O-acylated monophosphoryl lipid A 3D-MPL and QS21 in an oil/water emulsion.

3D-MPL: is a chemically detoxified form of the lipopolysaccharide (LPS) of the Gram-negative bacteria Salmonella minnesota.

Experiments performed at Smith Kline Beecham Biologicals have shown that 3D-MPL combined with various vehicles strongly enhances both the humoral and a TH1 type of cellular immunity.

QS21: is one saponin purified from a crude extract of the bark of the Quillaja Saponaria Molina tree, which has a strong adjuvant activity: it activates both antigen-specific lymphoproliferation and CTLs to several antigens.

Experiments performed at Smith Kline Beecham Biologicals have demonstrated a clear synergistic effect of combinations of 3D-MPL and QS21 in the induction of both humoral and TH1 type cellular immune responses.

The oil/water emulsion is composed of 2 oils (a tocopherol and squalene), and of PBS containing Tween 80 as emulsifier. The emulsion comprised 5% squalene 5% tocopherol 0.4% Tween 80 and had an average particle size of 180 nm (see WO 95/17210).

Experiments performed at Smith Kline Beecham Biologicals have proven that the adjunction of this O/W emulsion to 3D-MPL/QS21 further increases their immunostimulant properties.

Preparation of the Oil/Water Emulsion (2 Fold Concentrate)

Tween 80 is dissolved in phosphate buffered saline (PBS) to give a 2% solution in the PBS. To provide 100 ml two fold concentrate emulsion 5 g of DL alpha tocopherol and 5 ml of squalene are vortexed to mix thoroughly. 90 ml of PBS/Tween solution is added and mixed thoroughly. The resulting emulsion is then passed through a syringe and finally microfluidised by using an M110S microfluidics machine. The resulting oil droplets have a size of approximately 180 nm.

Preparation of Oil in Water Formulation.

Antigen prepared in accordance with example 1 or 2 (5 μg) was diluted in 10 fold concentrated PBS pH 6.8 and H₂O before consecutive addition of SB62, 3D-MPL (5 μg), QS21 (5 μg) and 50 μg/ml thiomersal as preservative at 5 min interval. The emulsion volume is equal to 50% of the total volume (50 μl for a dose of 100 μl).

All incubations were carried out at room temperature with agitation.

6. Immunogenicity of Tat and Nef-Tat in Rodents

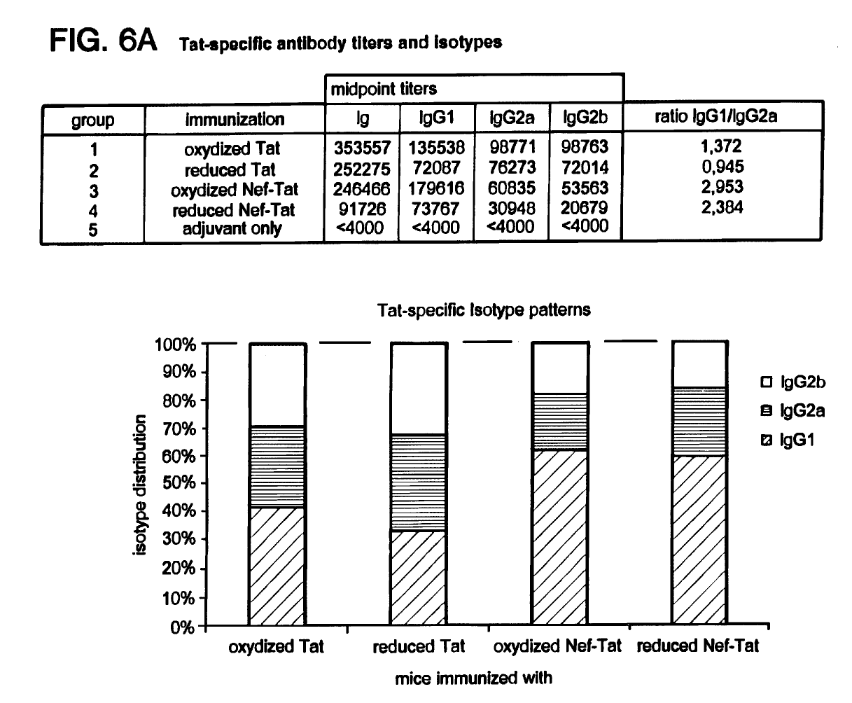

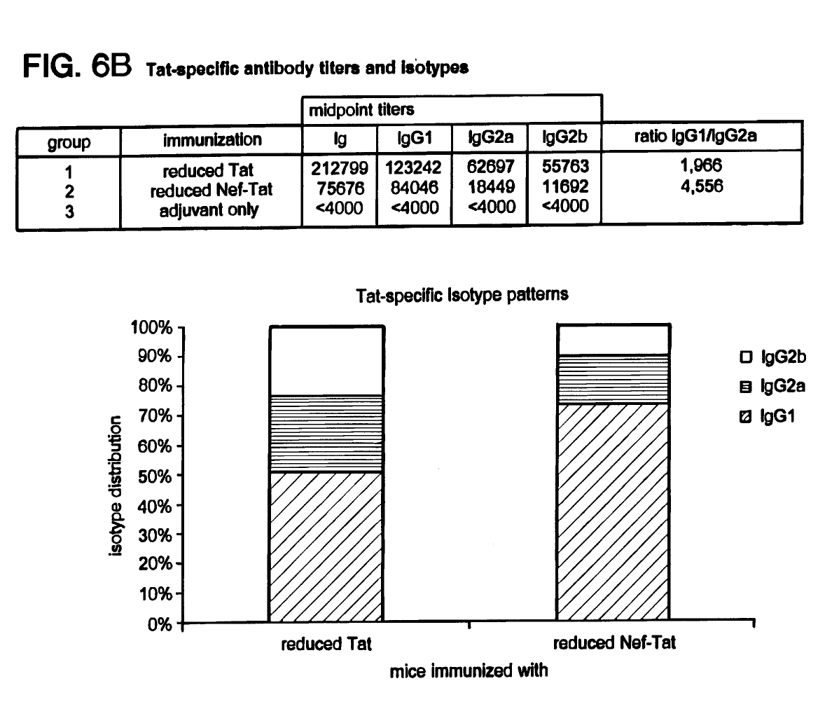

Characterization of the immune response induced after immunization with Tat and NefTat was carried out. To obtain information on isotype profiles and cell-mediated immunity (CMI) two immunization experiments in mice were conducted. In the first experiment mice were immunized twice two weeks apart into the footpad with Tat or NefTat in the oxydized or reduced form, respectively. Antigens were formulated in an oil in water emulsion comprising squalene, tween 80™ (polyoxyethylene sorbitan monooleate) QS21, 3D-MPL and □-tocopherol, and a control group received the adjuvant alone. Two weeks after the last immunization sera were obtained and subjected to Tat-specific ELISA (using reduced Tat for coating) for the determination of antibody titers and isotypes (FIG. 6a). The antibody titers were highest in the mice having received oxydized Tat. In general, the oxydized molecules induced higher antibody titers than the reduced forms, and Tat alone induced higher antibody titers than NefTat. The latter observation was confirmed in the second experiment. Most interestingly, the isotype profile of Tat-specific antibodies differed depending on the antigens used for immunization. Tat alone elicited a balanced IgG1 and IgG2a profile, while NefTat induced a much stronger T_{H2} bias (FIG. 6b). This was again confirmed in the second experiment.

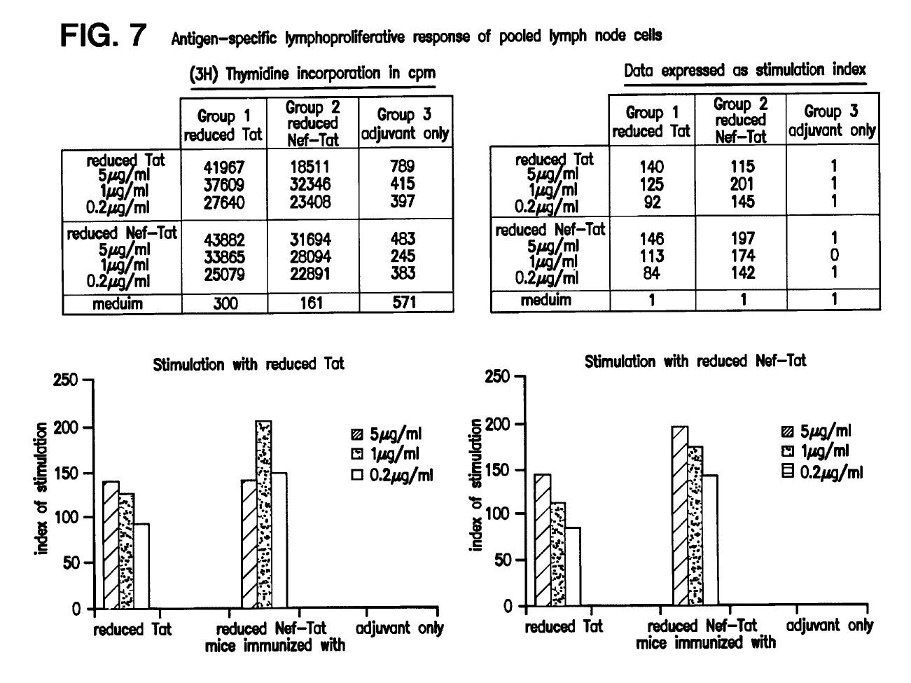

In the second mouse experiment animals received only the reduced forms of the molecules or the adjuvant alone. Besides serological analysis (see above) lymphoproliferative responses from lymph node cells were evaluated. After restimulation of those cells in vitro with Tat or NefTat ³H-thymidine incorporation was measured after 4 days of culture. Presentation of the results as stimulation indices indicates that very strong responses were induced in both groups of mice having received antigen (FIG. 7).

In conclusion, the mice studies indicate that Tat as well as Nef-Tat are highly immunogenic candidate vaccine antigens. The immune response directed against the two molecules is characterized by high antibody responses with at least 50% IgG1. Furthermore, strong CMI responses (as measured by lymphoproliferation) were observed.

7. Functional Properties of the Tat and Nef-Tat Proteins

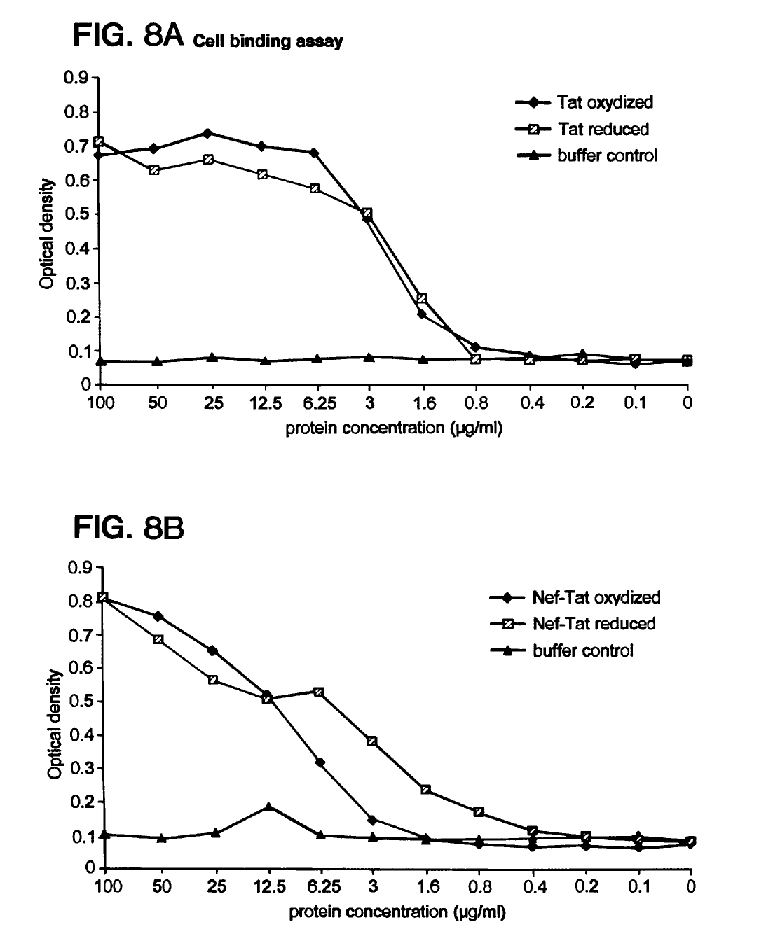

The Tat and NefTat molecules in oxydized or reduced form were investigated for their ability to bind to human T cell lines. Furthermore, the effect on growth of those cell lines was assessed. ELISA plates were coated overnight with different concentration of the Tat and NefTat proteins, the irrelevant gD from herpes simplex virus type II, or with a buffer control alone. After removal of the coating solution HUT-78 cells were added to the wells. After two hours of incubation the wells were washed and binding of cells to the bottom of the wells was assessed microscopically. As a quantitative measure cells were stained with toluidine blue, lysed by SDS, and the toluidine blue concentration in the supernatant was determined with an ELISA plate reader. The results indicate that all four proteins, Tat and NefTat in oxydized or reduced form mediated binding of the cells to the ELISA plate (FIG. 8). The irrelevant protein (data not shown) and the buffer did not fix the cells. This indicates that the recombinantly expressed Tat-containing proteins bind specifically to human T cell lines.

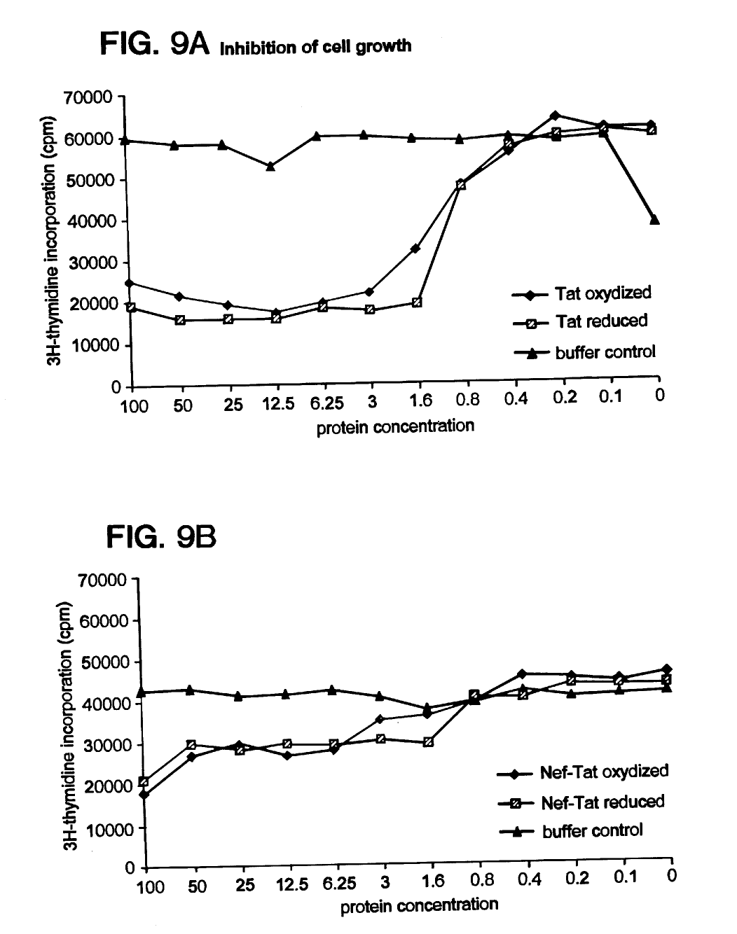

In a second experiment HUT-78 cells were left in contact with the proteins for 16 hours. At the end of the incubation period the cells were labeled with [³H]-thymidine and the incorporation rate was determined as a measure of cell growth. All four proteins included in this assay inhibited cell growth as judged by diminished radioactivity incorporation (FIG. 9). The buffer control did not mediate this effect. These results demonstrate that the recombinant Tat-containing proteins are capable of inhibiting growth of a human T cell line.

In summary the functional characterization of the Tat and NefTat proteins reveals that these proteins are able to bind to human Tcell lines. Furthermore, the proteins are able to inhibit growth of such cell lines.

1. An immunogenic composition comprising: a fusion protein comprising a polypeptide comprising amino acids 2-206 of HIV Nef linked to a polypeptide comprising amino acids 2-86 of HIV Tat, wherein the HIV Nef polypeptide and the HIV Tat polypeptide are linked in any orientation, and wherein the fusion protein is in admixture with a pharmaceutically acceptable excipient.

2. The immunogenic composition of claim 1, wherein the fusion protein comprises an entire HIV Nef protein, an entire HIV Tat protein, or an entire HIV Nef protein and an entire HIV Tat protein.

3. The immunogenic composition of claim 1, wherein the fusion protein comprises a polypeptide with a C-terminal histidine tail.

4. The immunogenic composition of claim 1, wherein one or both of the Nef polypeptide and the Tat polypeptide comprise a deletion, addition or substitution of one amino acid.

5. The immunogenic composition of claim 1, wherein the fusion protein comprises an HIV Tat polypeptide that bears an amino acid substitution of Alanine for Lysine at position 41 in the active site region, and amino acid substitutions of Lysine for Arginine at position 78 and Glutamic acid for Aspartic acid at position 80 in the RGD motif, wherein the amino acid positions are designated relative to SEQ ID NO: 11.

6. The immunogenic composition of claim 5, wherein the fusion protein comprises a Tat polypeptide comprising amino acids 2-86 of SEQ ID NO:23.

7. The immunogenic composition of claim 1, wherein the polypeptides of the fusion protein are linked in a Nef-Tat orientation.

8. The immunogenic composition of claim 1, wherein the polypeptides of the fusion protein are linked in a Tat-Nef orientation.

9. The immunogenic composition of claim 1, wherein the fusion protein further comprises an additional fusion partner polypeptide.

10. The immunogenic composition of claim 1, wherein the fusion partner polypeptide comprises about 100 amino acids from the N-terminal of Haemophilus influenza B protein D.

11. The immunogenic composition of claim 1, wherein the fusion protein is carboxymethylated.

12. The immunogenic composition of claim 1, further comprising an adjuvant.

13. The immunogenic composition of claim 12, wherein the adjuvant is a TH1 inducing adjuvant.

14. The immunogenic composition of claim 13, wherein the TH1 inducing adjuvant comprising monophosphoryl lipid A or a derivative thereof.

15. The immunogenic composition of claim 14, further comprising a saponin adjuvant.

16. The immunogenic composition of claim 15, further comprising an oil in water emulsion.

17. The immunogenic composition of claim 1, wherein the fusion protein is encapsulated in a liposome.

18. The immunogenic composition of claim 1, further comprising HIV gp160 or its derivative gp120.

19. The immunogenic composition of claim 1, wherein the fusion protein comprises an amino acid sequence selected from: SEQ ID NOs: 13, 17, 21 and 25.
